(19) United States
(12) Patent Application Publication
(10) Pub. No.: US 2019/0085312 A1
(43) Pub. Date: Mar. 21, 2019

(54) Theranostic Strategy for MMP-14-Expressing Glioblastomas Impacts Survival

(71) Applicant: The Board of Trustees of the Leland Stanford Junior University, Stanford, CA (US)

(72) Inventor: Heike E. Daldrup-Link, San Francisco, CA (US)

(21) Appl. No.: 16/192,508

(22) Filed: Nov. 15, 2018

Related U.S. Application Data
(63) Continuation-in-part of application No. 14/908,096, filed on Jan. 27, 2016.

(30) Foreign Application Priority Data
Aug. 2, 2013 (GB) 1313900.1

(51) Int. Cl. C12N 9/64; A61P 35/00; A61K 49/18; A61K 47/64

(52) U.S. Cl. CPC C12N 9/6491; A61K 47/64; A61K 49/1866; A61P 35/00

(57) ABSTRACT

A treatment method is provided in which glioblastoma is targeted by administering to a subject in an effective amount theranostic cross-linked iron oxide nanoparticles (CLIO) which are conjugated to a vascular disrupting agent (ICT) and secured with a matrix-metalloproteinase cleavable peptide.

### CROSS-REFERENCE TO RELATED APPLICATIONS

This application claims priority from U.S. Provisional Patent Application 62/586,712 filed Nov. 15, 2018, which is incorporated herein by reference.

This application is a Continuation-In-Part Application of U.S. application Ser. No. 14/908,096 filed on Jan. 27, 2016, which is incorporated herein by reference. U.S. application Ser. No. 14/908,096 filed on Jan. 27, 2016 is a 371 of PCT/EP2014/066087 filed Jul. 25, 2014, which is incorporated herein by reference. PCT/EP2014/066087 filed Jul. 25, 2014 claims priority to GB1313900.1 filed Aug. 2, 2013, which is incorporated herein by reference.

### STATEMENT OF GOVERNMENT SPONSORED SUPPORT

This invention was made with Government support under contract CA176519, CA190196, and CA199075 awarded by the National Institutes of Health. The Government has certain rights in the invention.

### FIELD OF THE INVENTION

The invention relates to treatment methods to target glioblastoma.

### BACKGROUND OF THE INVENTION

Glioblastoma multiforme (GBM) is the most frequent primary malignant brain tumor in adults and the leading cause of cancer-related death in children. The mean survival time is 12 months in both adults and children. GBM contains GBM-initiating cells (GIC) that play a central role in GBM development and disease recurrence. GICs possess enhanced self-renewal and invasive properties, promote tumor angiogenesis, and are resistant to the limited number of current therapies, notably temo-zolomide. Therefore, GICs represent the core problem of the dismal outcome of GBM. To achieve improved survival of GBM patients, novel therapeutic strategies are needed that target GICs. GICs are preferentially found in the perivascular niche and depend on tumor vessels for nutrition supply and survival. The efficacy of current GBM therapies with oral or intravenous drugs is hindered by their limited transendothelial permeability to the GIC niche. Previous drug-loaded nanocarrier systems relied on the enhanced permeability and retention (EPR) effect in tumors. However, the highly heterogeneous nature of the EPR effect can lead to poor delivery to the GIC niche and hence, poor therapeutic efficacy. A new, emerging strategy is to deliver vascular-disrupting agents (VDA), which do not rely on the EPR effect. VDAs target endothelial cells at the intraluminal surface of blood vessels, for example, by disrupting the colchi-cine-binding site of tubulin. This leads to vascular collapse and starvation of tumor cells supplied by these vessels, a very effective therapeutic strategy. In addition, VDAs selectively destabilize the tumor microvascular endothelial lining, causing a transient increase in vascular permeability and drug delivery to the perivascular tumor interstitium, the location of the GIC niche. Thus, the highly vascularized nature of GBMs and perivascular location of the GIC-vascular niche have spurred a lot of interest in VDAs. Previous VDAs, including combretastatin and 5,6-dimethylxanthenone-4-acetic acid, have led to significant necrosis in gliomas. However, the clinical efficacy of first-generation VDAs was limited by a high prevalence of cardiotoxicity.

### SUMMARY OF THE INVENTION

Glioblastoma (GBM) has a dismal prognosis. Evidence from preclinical tumor models and human trials indicates the role of GBM-initiating cells (GIC) in GBM drug resistance. With this invention a new treatment method is provided with tumor enzyme-activatable, combined therapeutic and diagnostic (theranostic) nanoparticles, which cause specific toxicity against GBM tumor cells and GICs. The theranostic cross-linked iron oxide nanoparticles (CLIO) were conjugated to a highly potent vascular disrupting agent (ICT) and secured with a matrix-metalloproteinase (MMP-14) cleavable peptide. Treatment with CLIO-ICT disrupted tumor vasculature of MMP-14-expressing GBM, induced GIC apoptosis, and significantly impaired tumor growth. In addition, the iron core of CLIO-ICT enabled in vivo drug tracking with MR imaging. Treatment with CLIO-ICT plus temozolomide achieved tumor remission and significantly increased survival of human GBM-bearing mice by more than 2-fold compared with treatment with temozolomide alone. Thus, a novel therapeutic strategy is provided with significant impact on survival and great potential for clinical translation.

### BRIEF DESCRIPTION OF THE DRAWINGS

Some of the figures are grey-scale conversion of color graphs. If needed, the reader is invited to U.S. Provisional Patent Application 62/586,712 filed Nov. 15, 2018, which is incorporated herein by reference, for reference to the original color graphs.

FIGS. 1A-I show according to an exemplary embodiment of the invention that CLIO-ICTs inhibit GBM and GIC survival in vitro. *, P<0.05; **, P<0.005, one-way ANOVA. DMSO, CLIO, ICT and CLIO-ICT bars in FIGS. 1D-F, and 1I are from left to right in each column pair.

FIGS. 2A-E show according to an exemplary embodiment of the invention that CLIO-ICTs retard GBM growth in vivo. *, P=0.0002 and *, P=0.0003 for pcGBM39 (n=8) and pcGBM2 (n=6), respectively, one-way ANOVA. E, Kaplan-Meier survival curves of control and treated mice demonstrate a significant survival benefit of CLIO-ICT as compared with vehicle, log-rank Mantel-Cox test.

FIGS. 3A-E show according to an exemplary embodiment of the invention CLIO-ICTs delivered to GBM tumors and induce tumor apoptosis. (*, P<0.05; **, P<0.005, n=6, one-way ANOVA).

FIG. 4 shows according to an exemplary embodiment of the invention that CLIO-ICTs target GBM vasculature and GICs in vivo. Results are representative of three independent experiments (*, P<0.05; **, P<0.005, one-way ANOVA).

FIGS. 5A-D show according to an exemplary embodiment of the invention that CLIO-ICTs induce GIC apoptosis in vivo. (P<0.05; **, P<0.005, n=6, one-way ANOVA).

FIG. 6A-I show according to an exemplary embodiment of the invention that CLIO-ICTs increase the antitumor efficacy of temozolomide (TMZ) in vivo. *, P<0.05, respectively, n=6 (pcGBM39) and n=6 (pcGBM2), one-way ANOVA. FIGS. 6F, 6I show Kaplan-Meier survival curves of control and treated mice (n=6, pcGBM39; n=6, pcGBM2) demonstrating a significant survival benefit of CLIO-ICT and ICT in combination with TMZ as compared with vehicle, log-rank Mantel-Cox test (*, P<0.05; **, P<0.005, n=6, one-way ANOVA).

### DETAILED DESCRIPTION

To avoid concomitant toxic effects in normal organs, nontoxic VDA-prodrugs can be designed, which are activated by specific tumor enzymes. For example, matrix metalloproteinases (MMP) and specifically the membrane-type MMPs (MT-MMPs; MT1-MMP 1/4 MMP-14) subclass represent an ideal target for prodrug activation, because they are highly overexpressed in GBM and can selectively cleave specific peptide sequences. The azademethylcolchicine-peptide conjugate ICT2588 is metabolized by MMP-14 to release an active VDA, azademethylcolchicine, with efficacy against a range of solid tumors and minimal systemic toxicity. We coupled ICT, a minor structural analogue of ICT2588 (modified to allow conjugation to nanoparticles), to cross-linked iron oxide nano-particles (CLIO) to generate theranostic nanoparticles (CLIO-ICT). Feasibility has been shown for MMP-14-specific cleavage, efficient drug delivery, and therapeutic efficacy in murine mammary adenocarcinomas. This new theranostic strategy should be particularly beneficial for GBM, where efficient drug treatment is limited by the blood—brain barrier and inability to reach the perivascular GIC niche. On the basis of the vascular disrupting properties of the ICT drug, CLIO-ICT should be able to permeate the tumor microvascular endothelium and reach tumor cells and GICs. In this invention, I show in an exemplary embodiment that CLIO-ICT induces significant apoptosis of GBM tumor cells and GIC, and thereby, prolong survival of GBM-bearing mice. Embodiments of the invention are not limited to mice, but extent to other animals and humans. Embodiments of CLIO-ICT and methods of making CLIO-ICTs used for the methods described herein the reader is referred to U.S. application Ser. No. 14/908,096 filed on Jan. 27, 2016, which is incorporated herein by reference for all that it teaches. The CLIO-ICTs used herein as the same or similar to the ones used in U.S. application Ser. No. 14/908,096. For specific details pertaining materials and methods used in experiments, verification protocols, or the like related to the method of this invention, the reader is also referred to Appendix A in U.S. Provisional Patent Application 62/586,712 filed Nov. 15, 2018, which is incorporated herein by reference for all that is teaches.

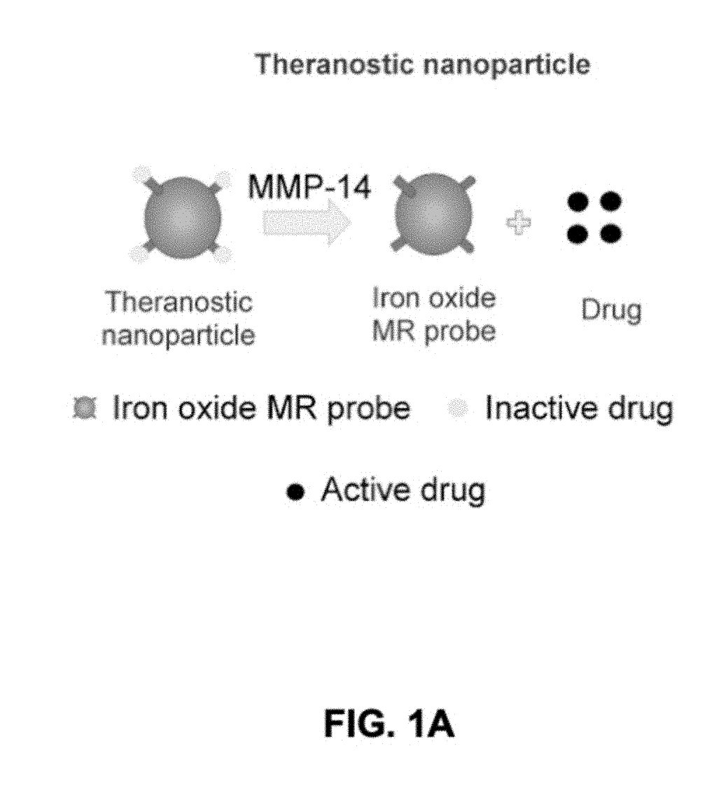

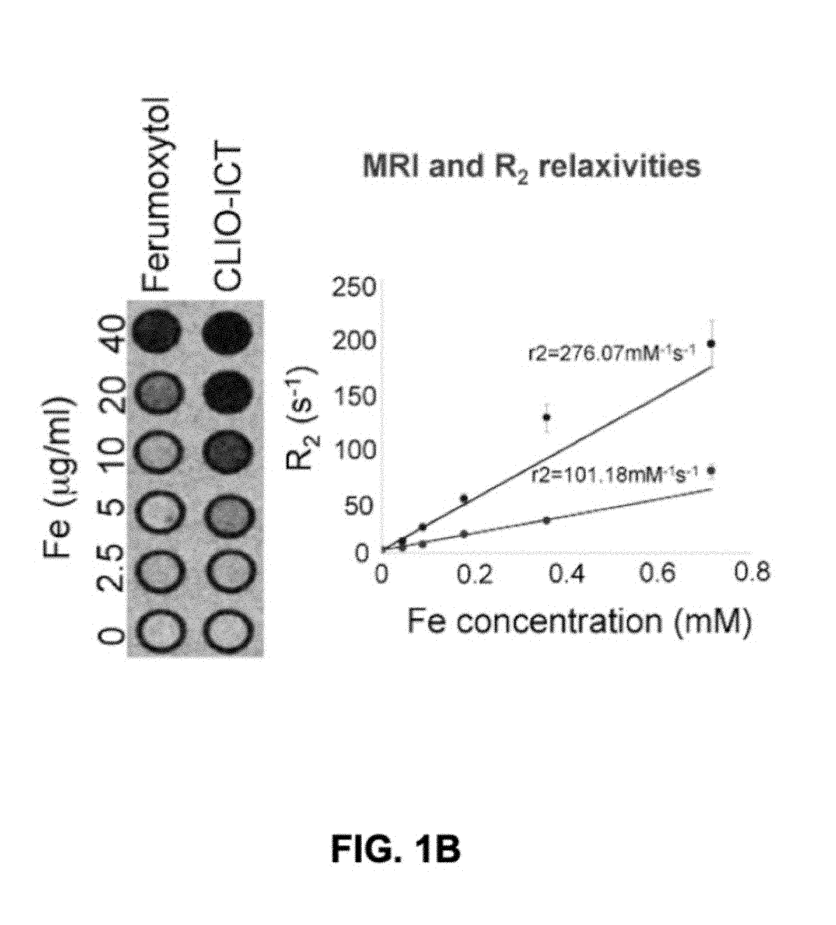

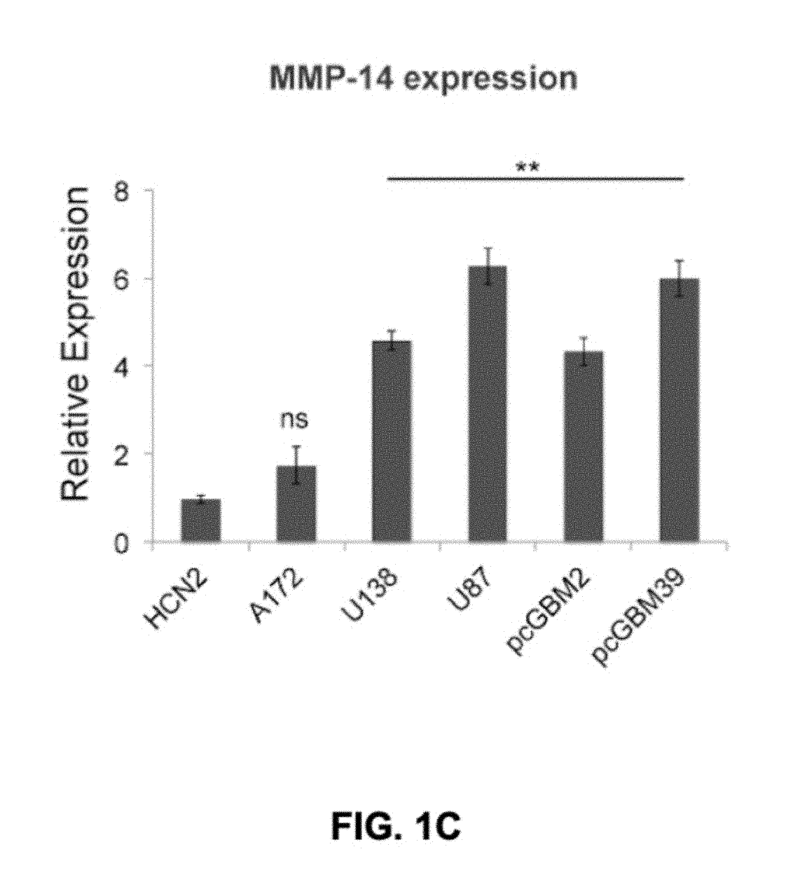

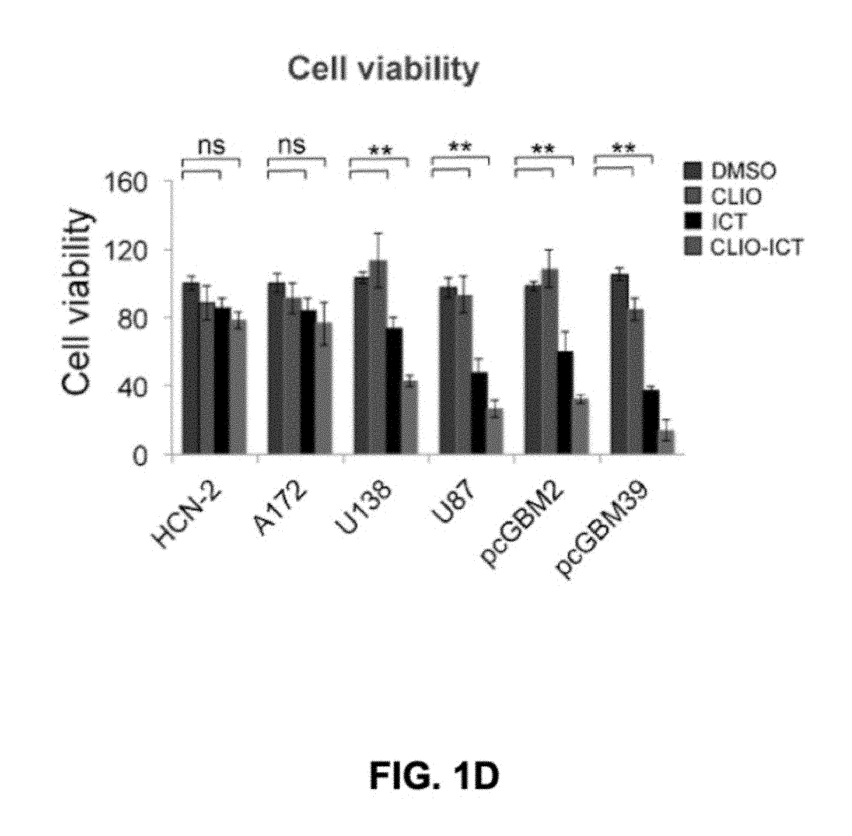

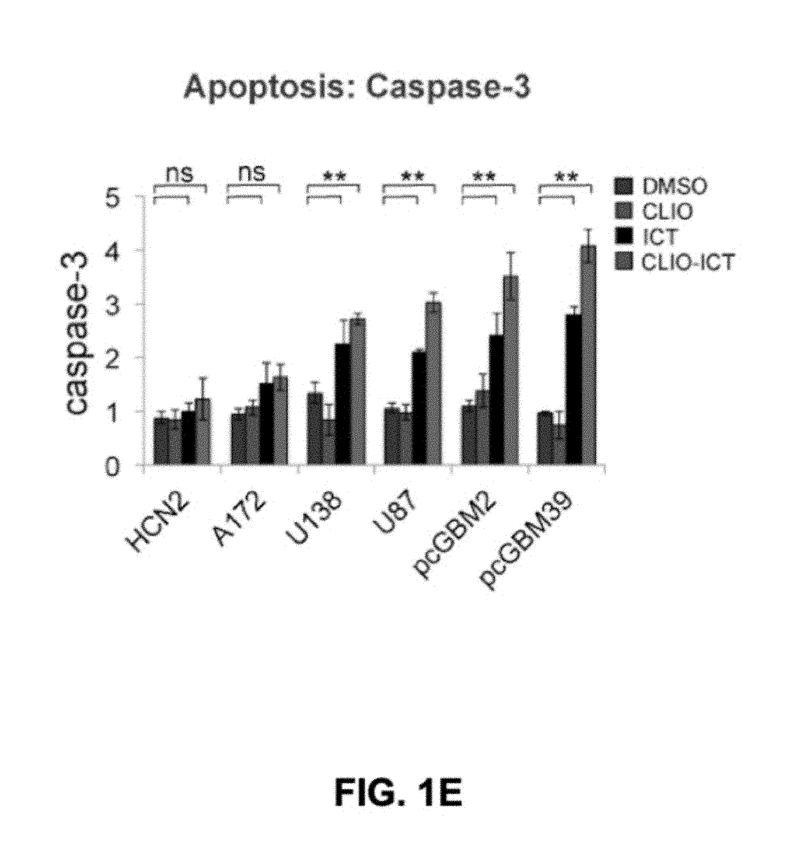

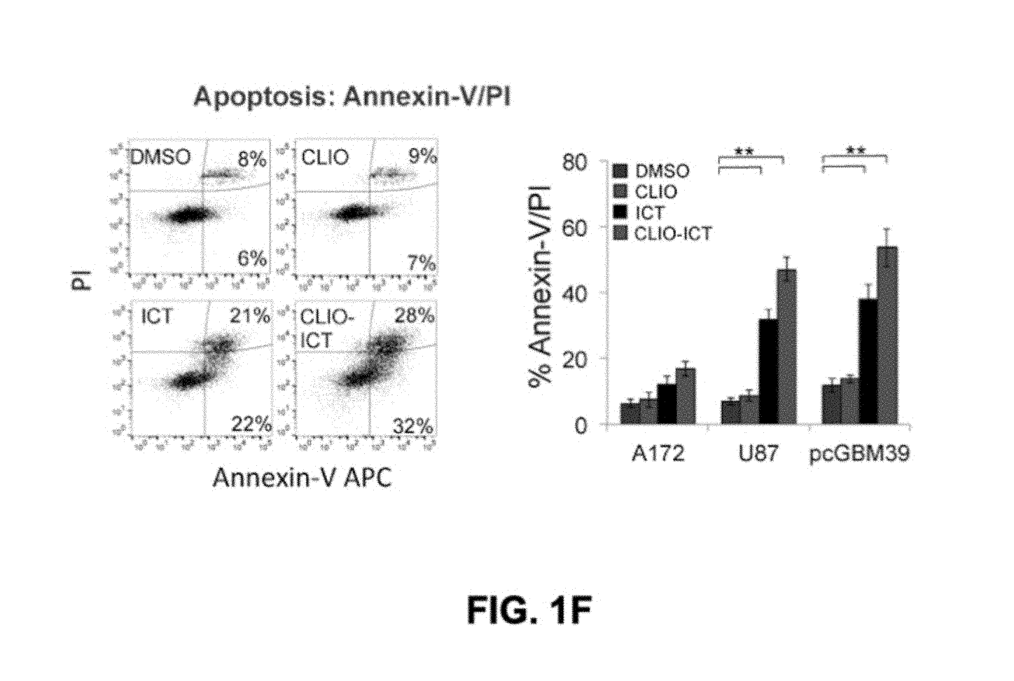

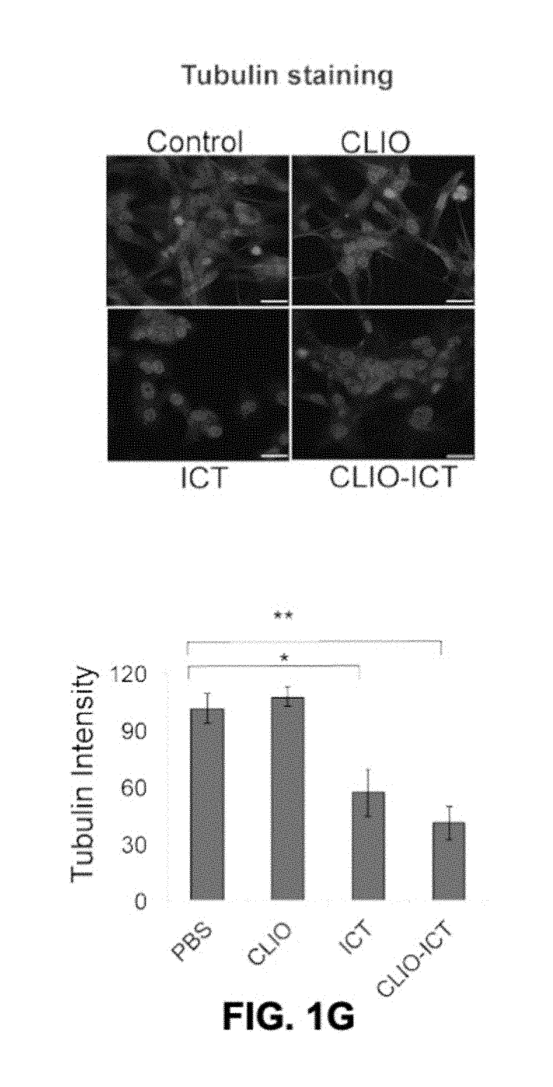

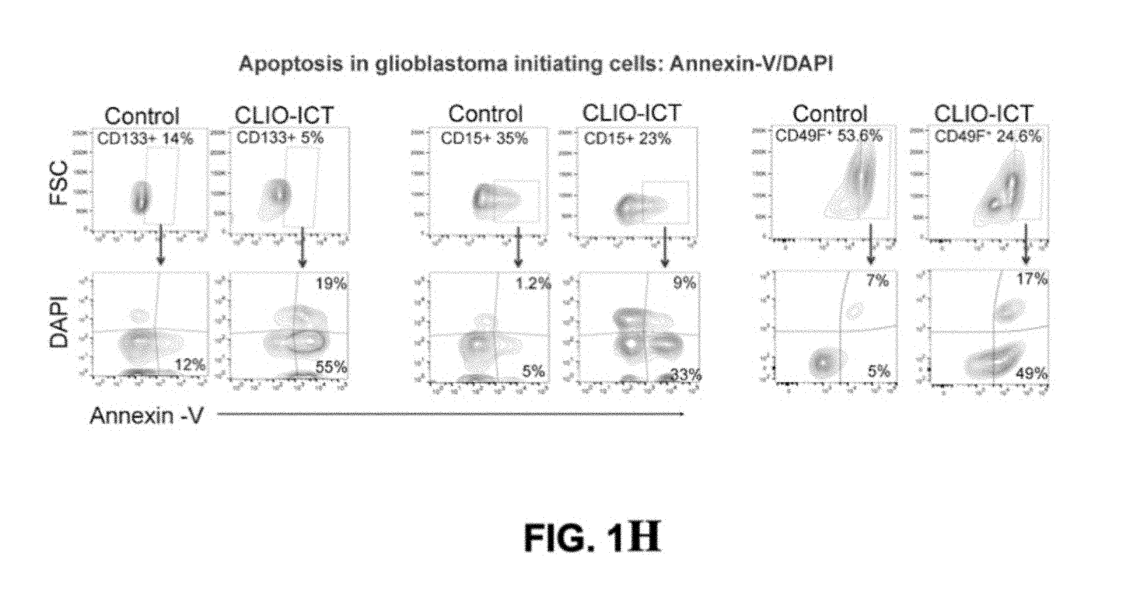

FIGS. 1A-I show CLIO-ICTs inhibit GBM and GIC survival in vitro. The concept for MMP-14 activatable theranostic nanoparticles, CLIO-ICTs in GBMs is shown in FIG. 1A. FIG. 1A shows a schematic demonstration of CLIO-ICT activation in presence of tumor enzyme MMP-14, to release the active drug, azademethylcolchicine. Azademethylcolchicine targets tubulin to induce apoptosis in tumor cells. ICT and CLIO-ICT were prepared and characterized as described by Ansari (Ansari et al. Development of novel tumor-targeted theranostic nanoparticles activated by membrane-type matrix metalloproteinases for combined cancer magnetic resonance imaging and therapy. Small 2014, 10:566-75). The hydrodynamic diameter of CLIO-ICT nanoparticles, as measured with a Zetasizer Nano ZS analyzer, was 22.10±0.78 nm, slightly larger than ferumoxytol nanoparticles (20±1.02 nm). The average number of ICT molecules per iron oxide nanoparticle was 4.7, based on the attached fluorescein absorption and known CLIO-ICT concentration.

FIG. 1B shows representative T₂-weighted MR images of CLIO-ICT and ferumoxytol at different dilutions. T₂ MSME sequences were used to generate R₂ relaxivites. R₂ relaxivities of CLIO-ICT (blue line) and ferumoxytol (red line). CLIO-ICTs had higher r₂ relaxation rates (276.07 mmol/L⁻¹s⁻¹) compared with original ferumoxytol nanoparticles (101.18 mmol/L⁼¹s⁻¹, respectively; FIG. 1B). This is likely due to the modified surface coating.

FIG. 1C shows a graphical representation of MMP-14 expression in different GBM cells lines. MMP-14 expression was measured with qPCR assay, GAPDH served as endogenous control. FIG. 1D shows a viability analysis of GBMs treated with CLIO-ICT (10 nmol/L), ICT (10 nmol/L), CLIO (0.01 mmol/L), and PBS. GBMs were treated for 96 hours and viability was assayed using MTS assay.

FIG. 1E shows a fold change for cleaved caspase-3 in control and treated GBM cells. Cleaved caspase-3 fluorescence signals were detected by SensoLyte Homogeneous AMC Caspase-3/7 assay kit and the fold change was represented by the ratio: fluorescence signals in treated/fluorescence signals in control.

FIG. 1F shows Annexin-V/PI apoptosis staining in control and treated GBM39 cells (left). Right, quantification for percentage apoptosis in A172, U87 and pcGBM39 cells after 48 hours of treatment.

FIG. 1G shows immunofluorescence for a-tubulin in control and treated U87 cells. Top, confocal images and lower panel depicts quantification of tubulin signals (scale bar, 10 mm).

FIG. 1H shows a flow cytometry analysis of GIC markers (CD133, CD15, and CD49F), Annexin V/DAPI staining in control and treated pcGBM39 cells. CLIO-ICT-treated pcGBM39 were subjected to flow cytometric staining for GIC surface markers (top) and apoptosis markers (bottom).

FIG. 1I shows percentages of CD133⁺, CD15⁺, and CD49F⁺GICs (left) and percentage apoptosis (right) in gated CD133⁺, CD15+, and CD49F+ GICs from control and treated pcGBM39 cells. Results are represented as mean & SD from three independent experiments.

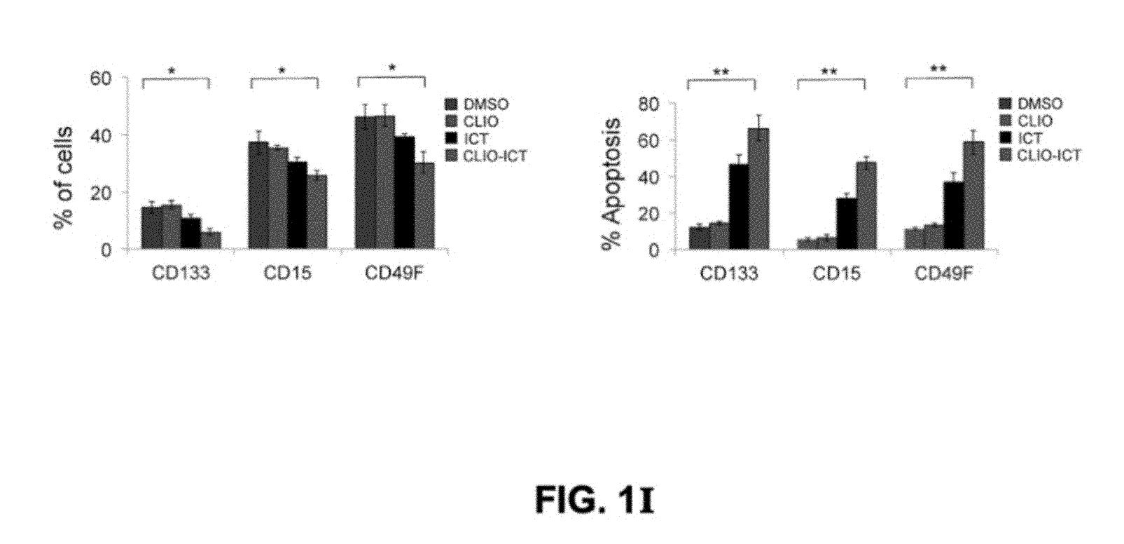

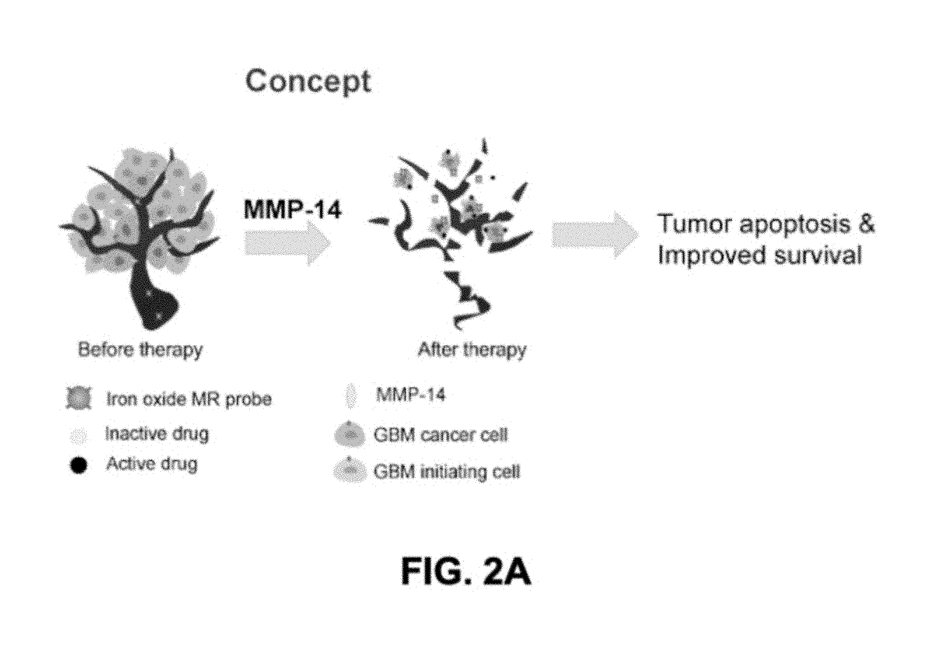

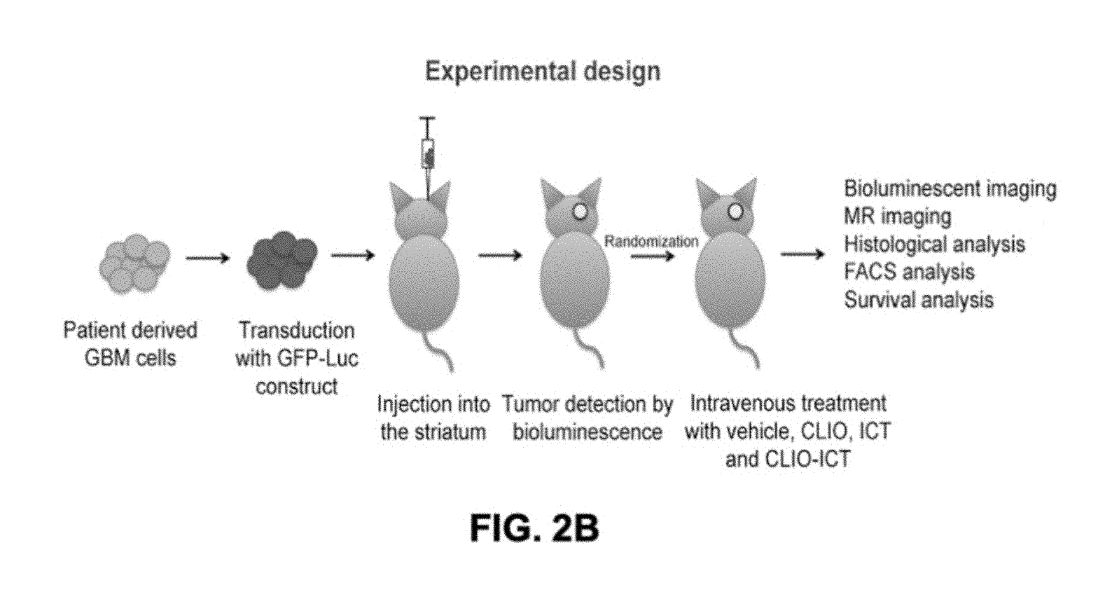

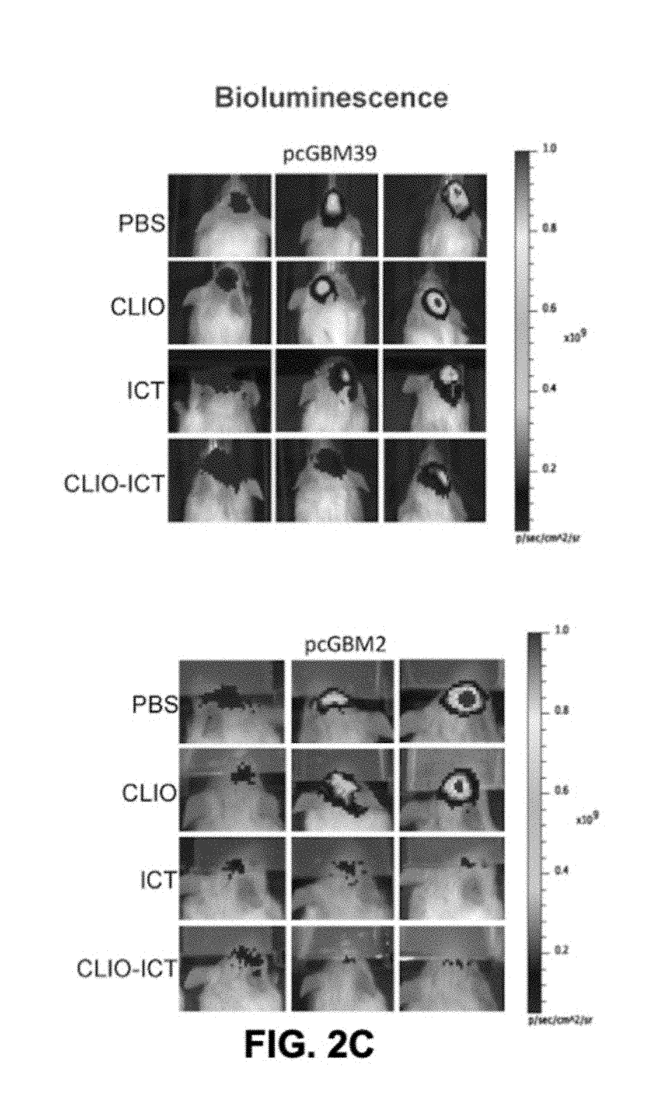

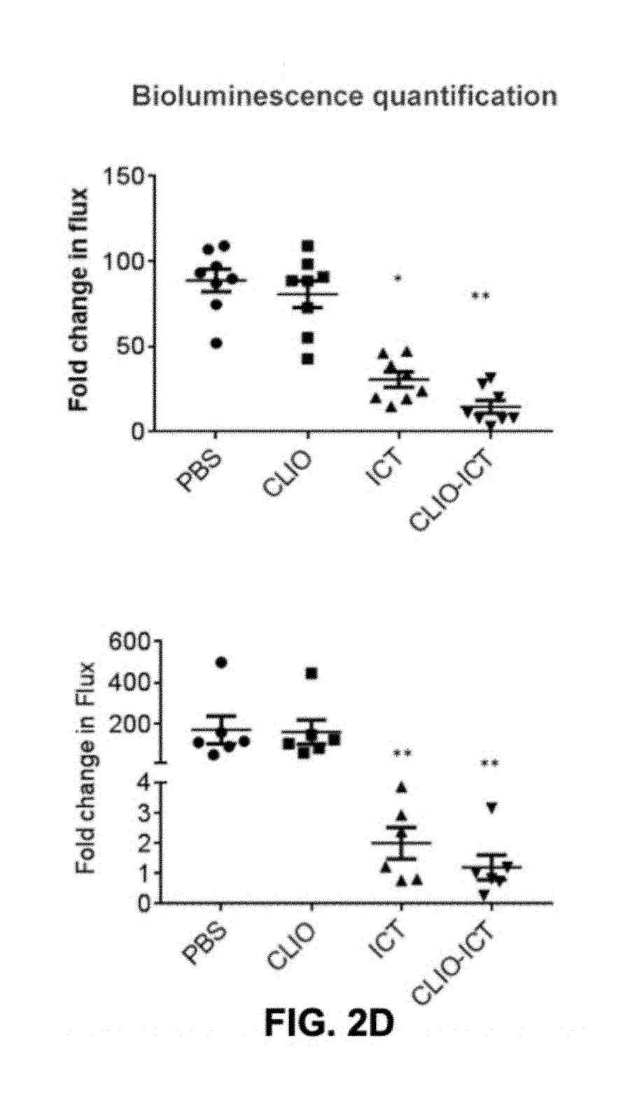

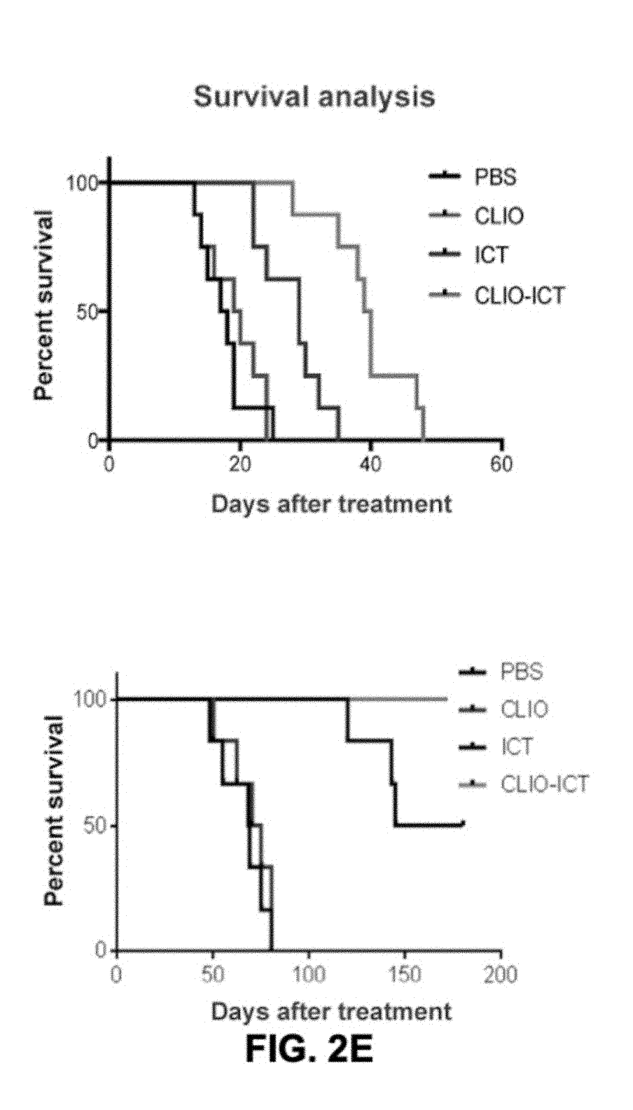

FIGS. 2A-E show that CLIO-ICTs retard GBM growth in vivo. FIG. 2A shows a schematic demonstration of CLIO-ICT-mediated anti-GBM effect. In presence of tumor enzyme MMP-14, CLIO-ICT is cleaved to release the active vasculature-disrupting agent, azademethylcolchicine. Activated CLIO-ICT targets tumor vasculature and induces apoptosis in GICs and GBMs, thereby inhibiting GBM growth and improving survival outcomes.

FIG. 2B shows a schematic representation of experimental design. Tumors were initiated with orthotopic injections of primary human GBM samples (pcGBM39 cells and pcGBM2 cells that express luciferase and GFP-luciferase construct, respectively) into the striatum of NSG mice. Treatment was initiated once the tumors were detected and bioluminescent analyses were performed during and after treatment.

FIG. 2C shows bioluminescent in vivo images of tumors in mice treated with CLIO-ICT (0.5 mmol Fe/kg and 80 mg/kg of ICT), ICT (80 mg/kg of ICT), CLIO (0.5 mmol Fe/kg), or vehicle. D, Quantification of the bioluminescent signals. Fold change in total flux represents the ratio: total flux after treatment/total flux before treatment.

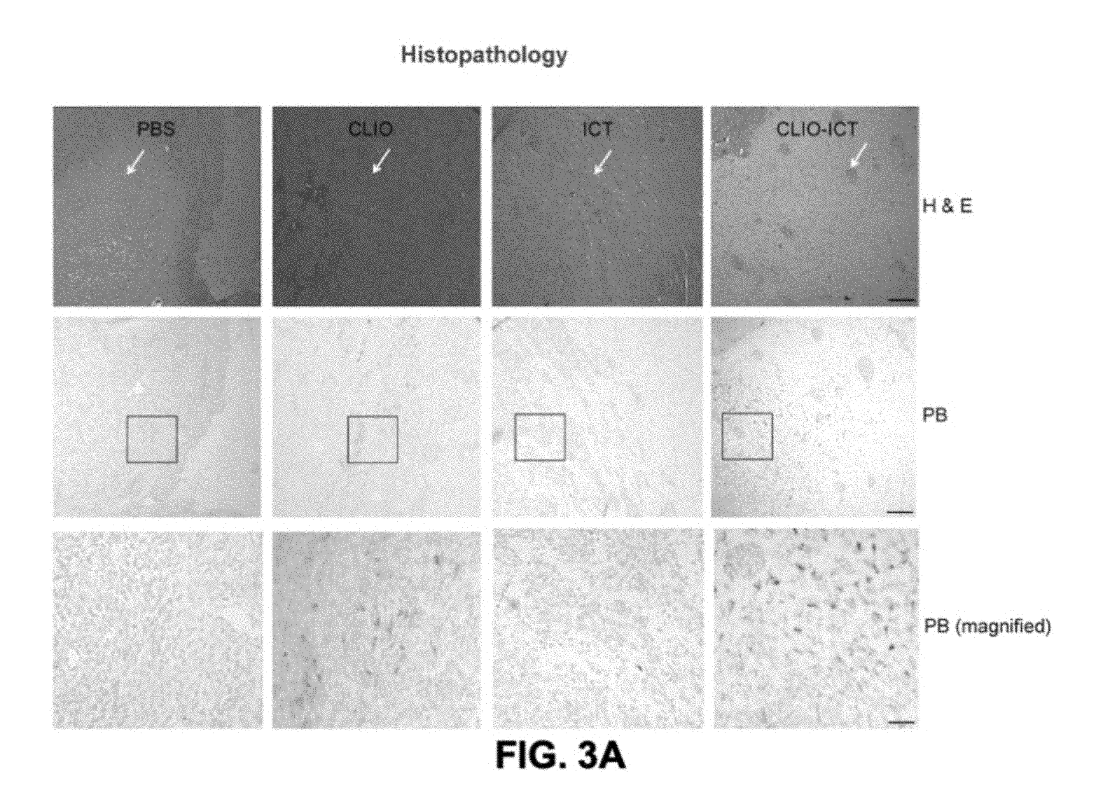

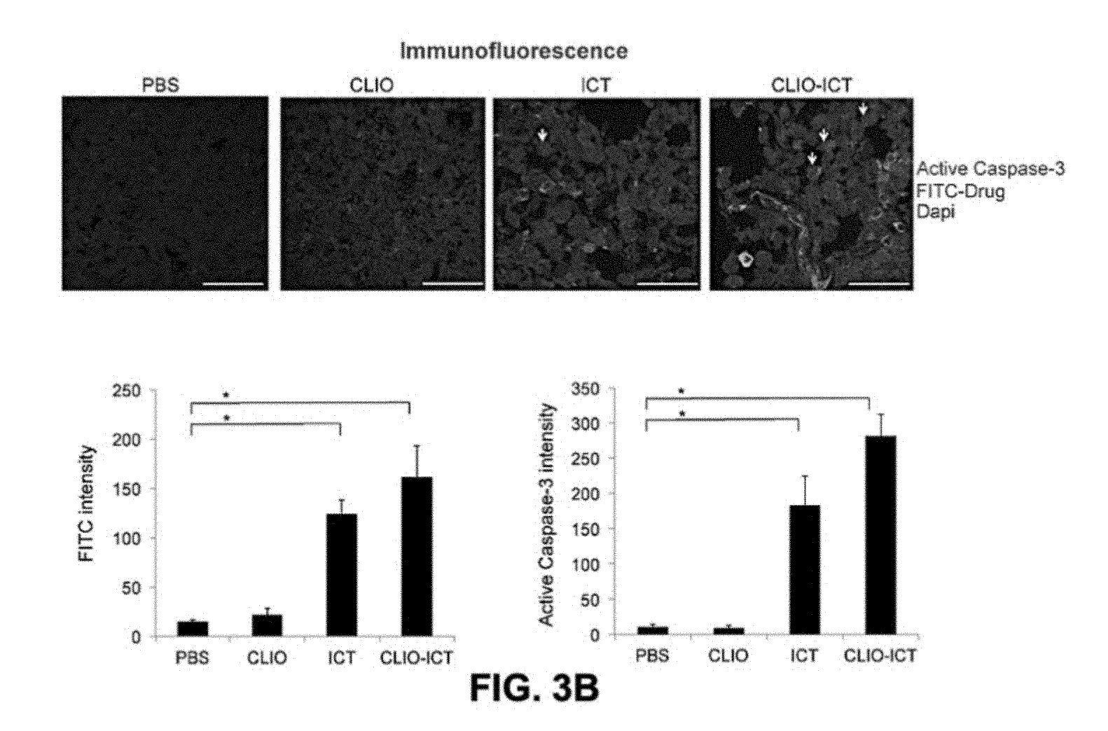

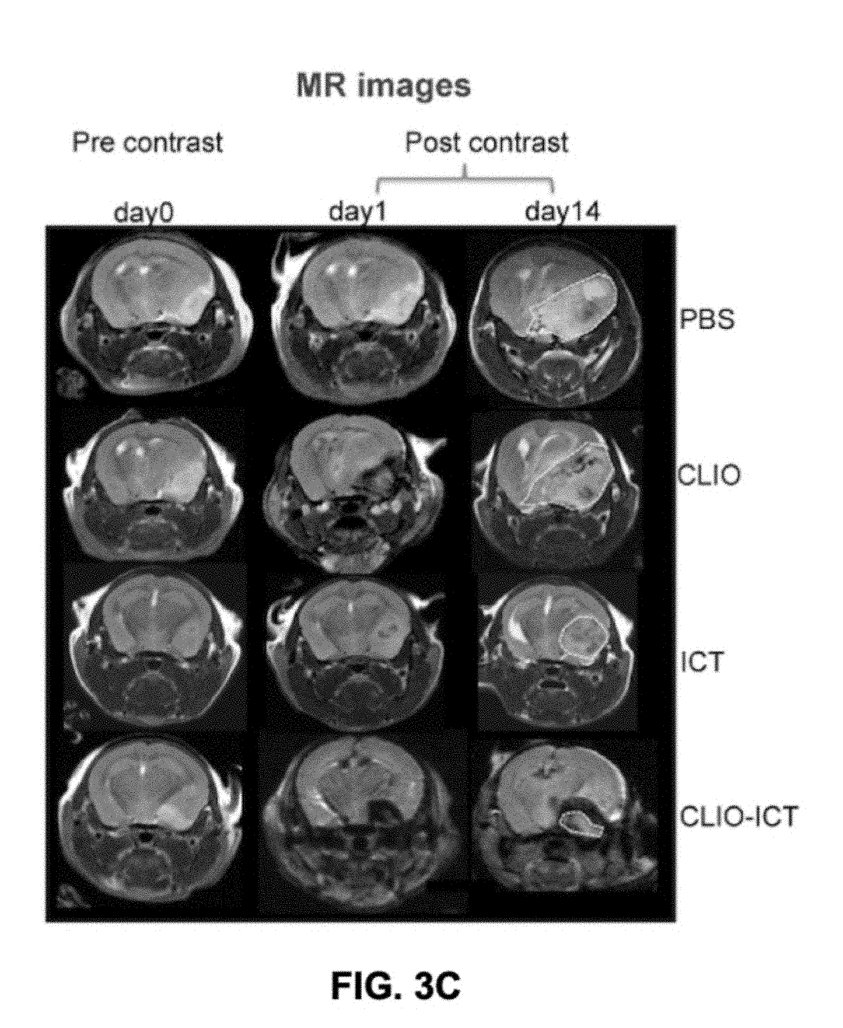

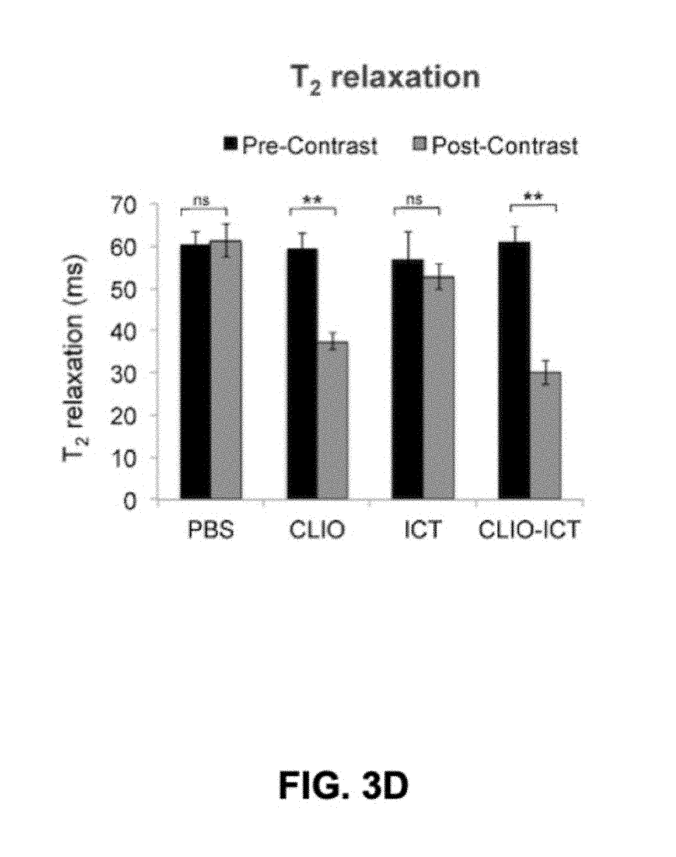

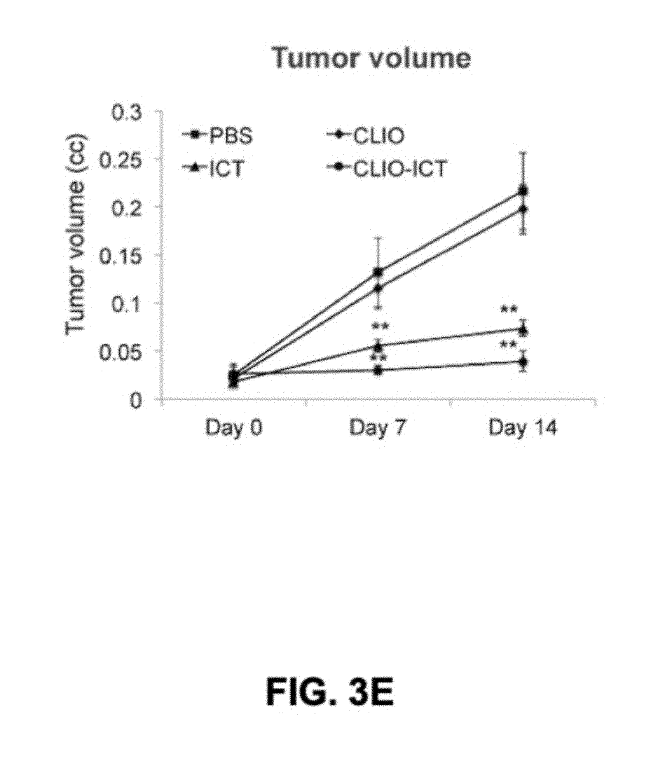

FIGS. 3A-E shows CLIO-ICTs delivered to GBM tumors and induce tumor apoptosis. Tumors were initiated with orthotopic injections of pcGBM39 tumors and mice were treated with CLIO-ICT (0.5 mmol Fe/kg and 80 mg/kg of ICT), ICT (80 mg/kg of ICT), CLIO (0.5 mmol Fe/kg) or vehicle.

FIG. 3A, Top shows representative H&E staining for brain coronal sections from control and treated animals. White arrows indicate tumor. Objective 4#. Scale bar, 100 mm. Middle, Prussian blue iron staining for control and treated tumors. Objective 4#. Scale bar, 100 mm. FIG. 3A, Bottom, Prussian blue at higher magnification 10# for boxed regions in middle panel. Scale bar, 40 mm.

FIG. 3B shows representative immunofluorescence confocal images for cleaved caspase-3 in control and treated pcGBM39 tumors. Arrows indicate cleaved caspase-3—positive cells. Nuclei counterstained with DAPI. Scale bar, 75 mm. Graph shows quantification for FITC and cleaved caspase-3 staining in control and treated pcGBM39 tumors. Results are represented as mean & SD from three independent experiments.

FIG. 3C shows representative T₂-weighted MR images of mice brain. T₂ FSE sequences were used to capture coronal T₂-weighted images. Nanoparticle and theranostic nanoparticle delivery is demonstrated by T₂ darkening or negative enhancement in CLIO and CLIO-ICT—treated animals, respectively. On day 14, tumor periphery is marked by dotted yellow line.

FIG. 3D shows quantification of T₂ darkening. T₂ MSME sequences were used to generate T₂ maps, Osirix software was used to calculate T₂ values. CLIO and CLIO-ICT-treated tumors demonstrated shorter T₂ values corresponding to T₂ darkening or negative enhancement.

FIG. 3E shows quantification of tumor volumes before and after treatment. T₂-weighted MR scans were used to calculate tumor volumes using Osirix software.

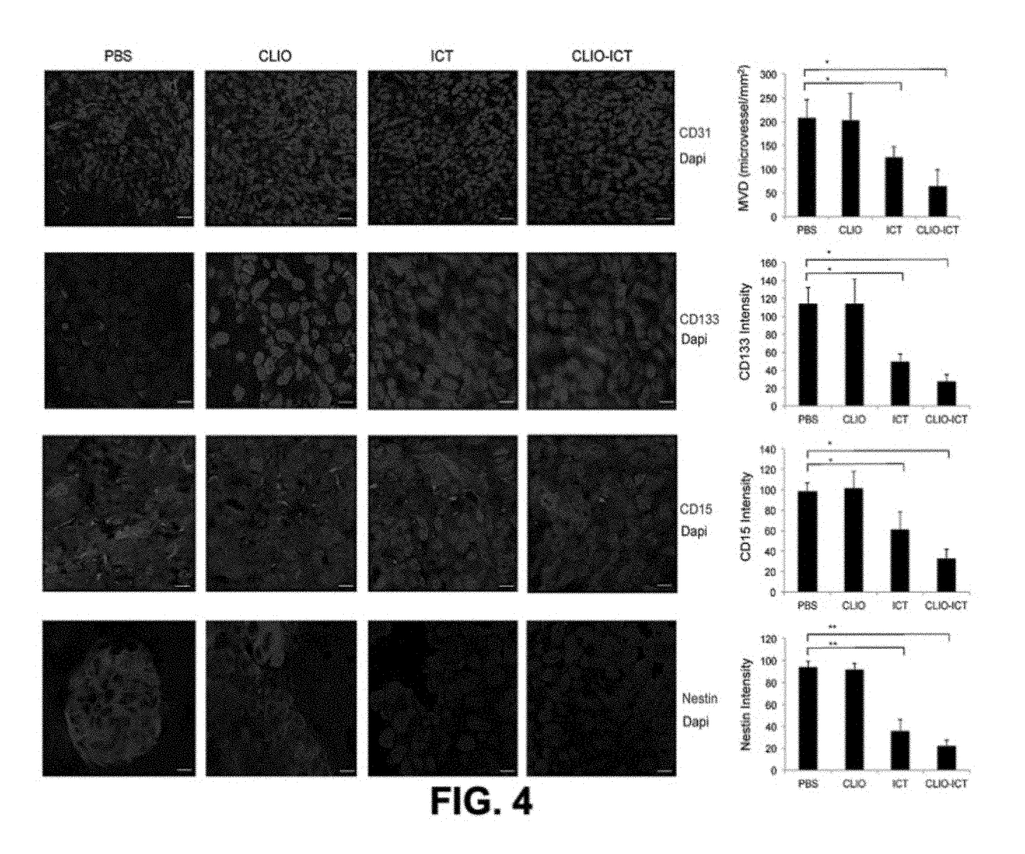

FIG. 4 shows CLIO-ICTs target GBM vasculature and GICs in vivo. Tumors were established with orthotopic injections of pcGBM39 cells. Mice were treated with CLIO-ICT (0.5 mmol Fe/kg and 80 mg/kg of ICT), ICT (80 mg/kg of ICT), CLIO (0.5 mmol Fe/kg), or vehicle. Representative immunofluorescent confocal images depicting CD31, CD133, CD15, and Nestin staining in pcGBM39 tumors. CD31 is used to outline tumor vasculature. CD133, CD15, and nestin are used to mark GICs. Signal intensity for CD31, CD133, CD15, and nestin in control and treated tumors have been quantified and represented graphically. Scale bar, 20 mm (CD31) and 10 mm (CD133, CD15 and nestin).

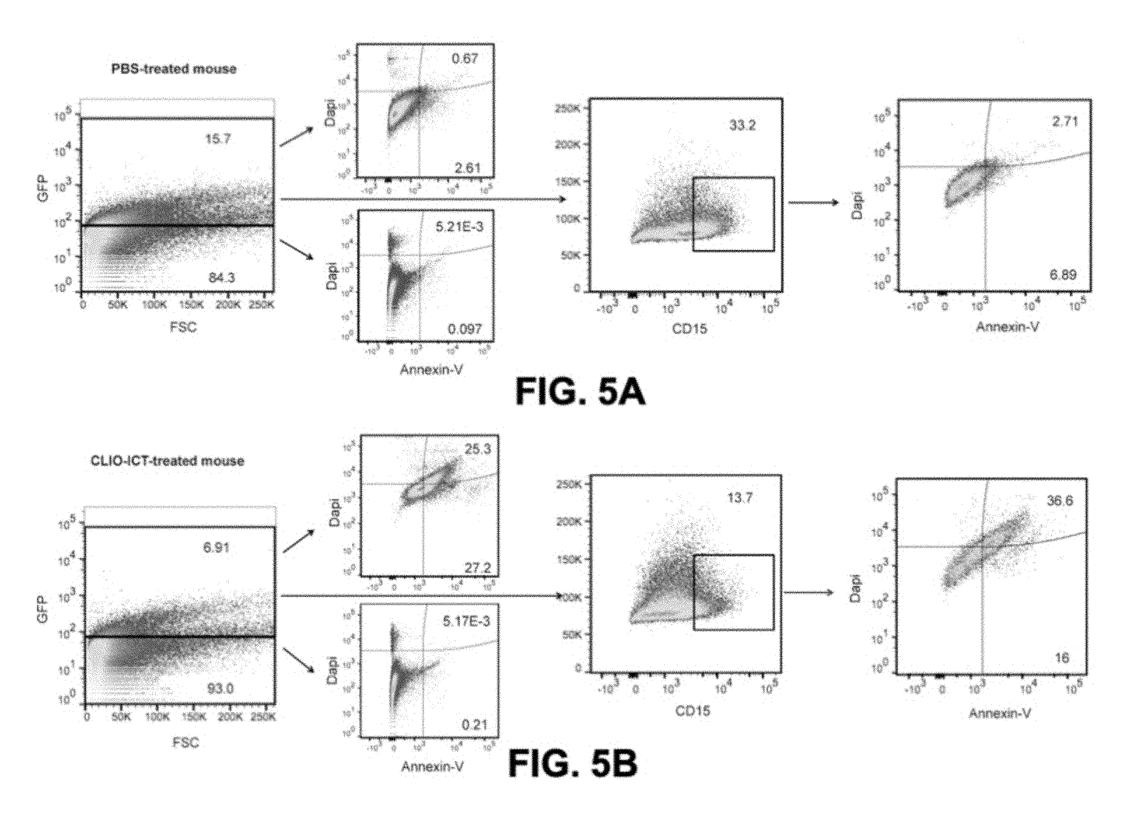

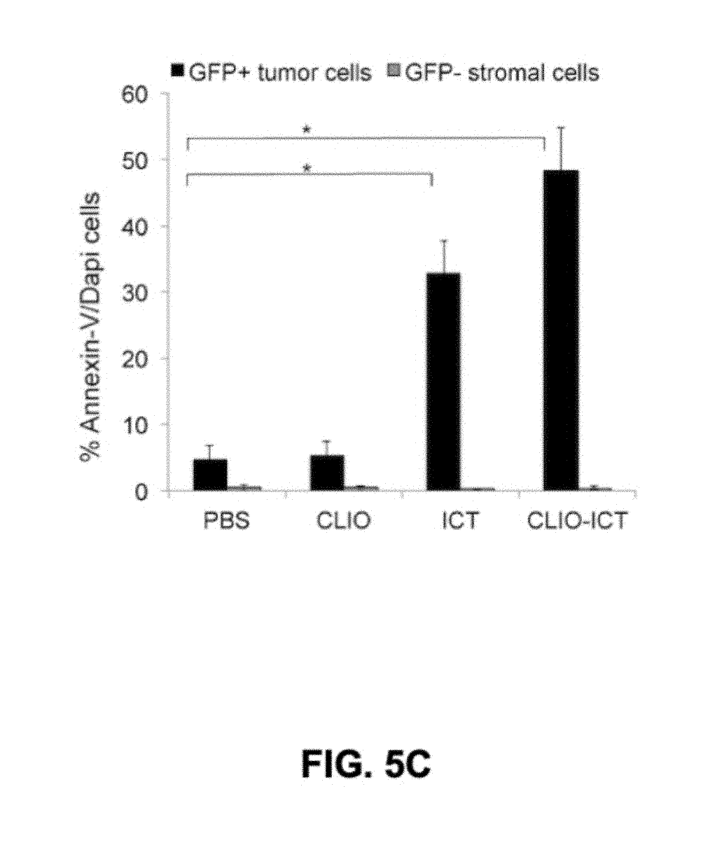

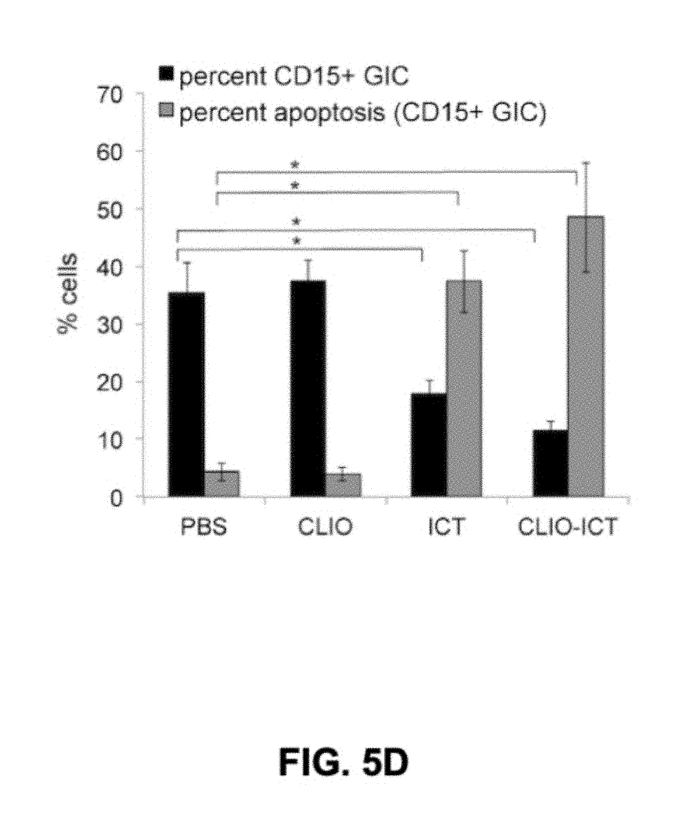

FIGS. 5A-D show CLIO-ICTs induce GIC apoptosis in vivo. Tumors were initiated with orthotopic injections of pcGBM2 cells. Mice were treated with CLIO-ICT (0.5 mmol Fe/kg and 80 mg/kg of ICT), ICT (80 mg/kg of ICT), CLIO (0.5 mmol Fe/kg), or vehicle. Flow cytometric analysis of GIC markers (CD15), Annexin V, and DAPI in (FIG. 5A) vehicle and (FIG. 5B) CLIO-ICT-treated pcGBM2 tumors. GFP-positive pcGBM2 tumors were gated and analyzed for percentage apoptosis in both pcGBM2 cells (FIG. 5C) and GICs from pcGBM2 (FIG. 5D).

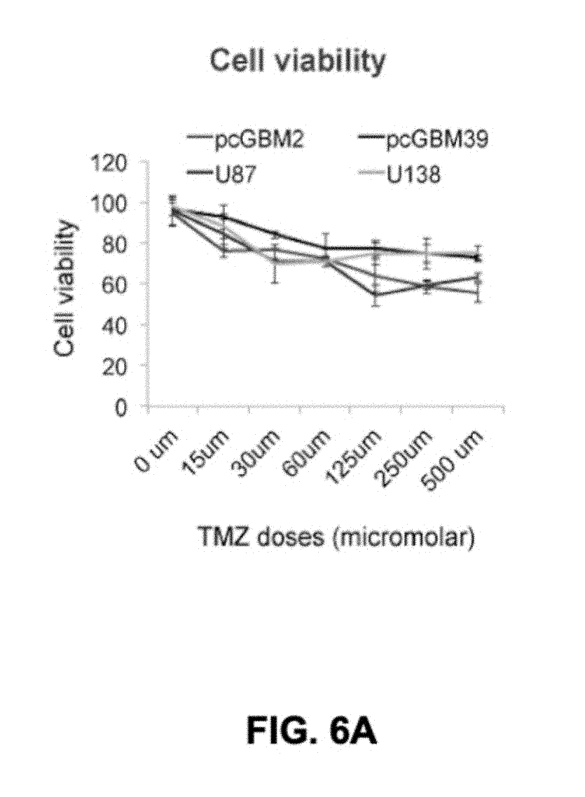

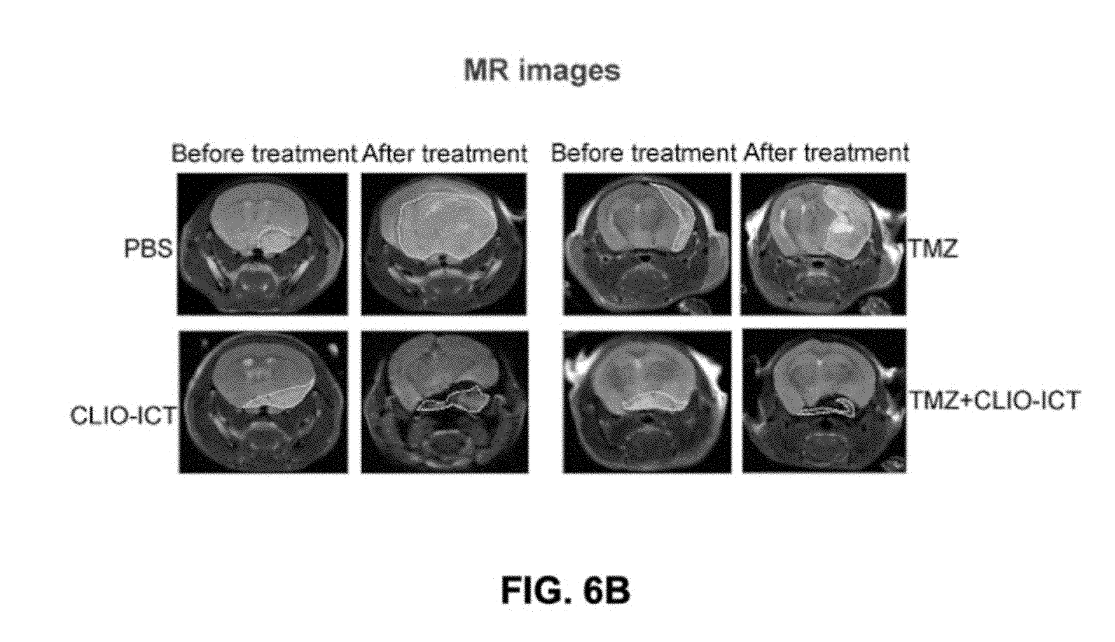

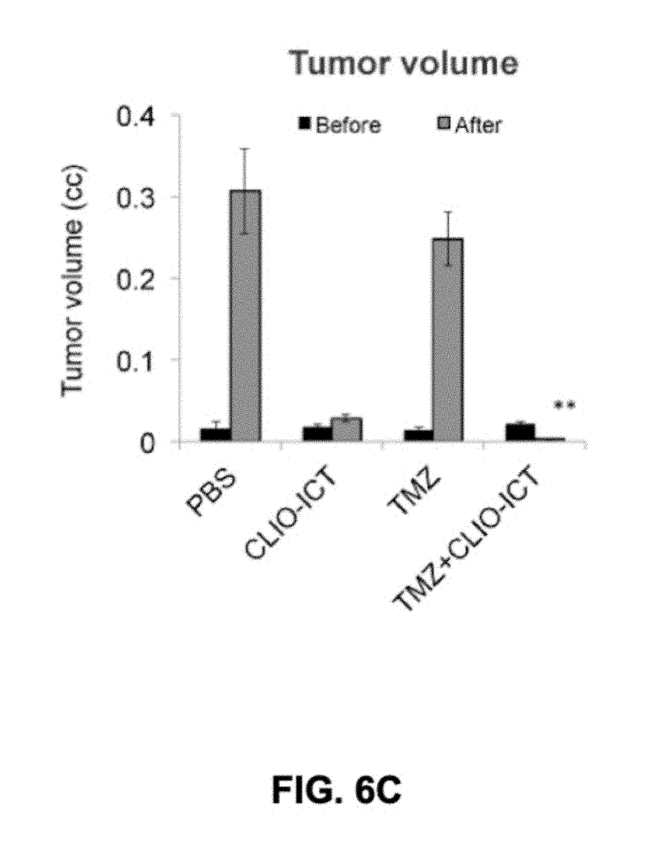

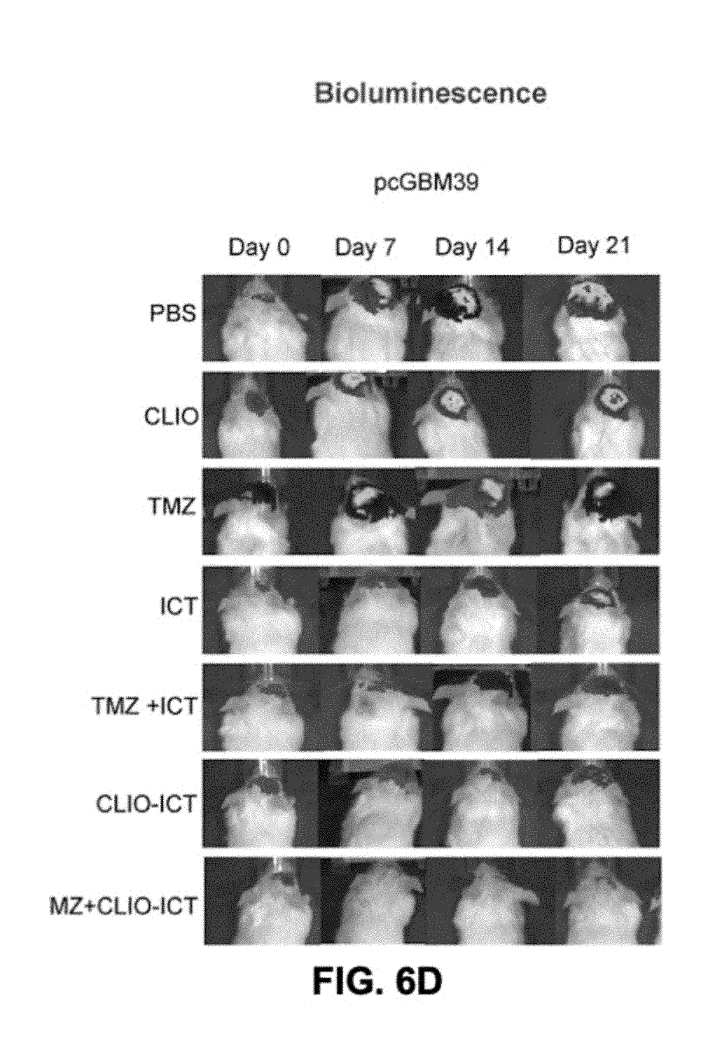

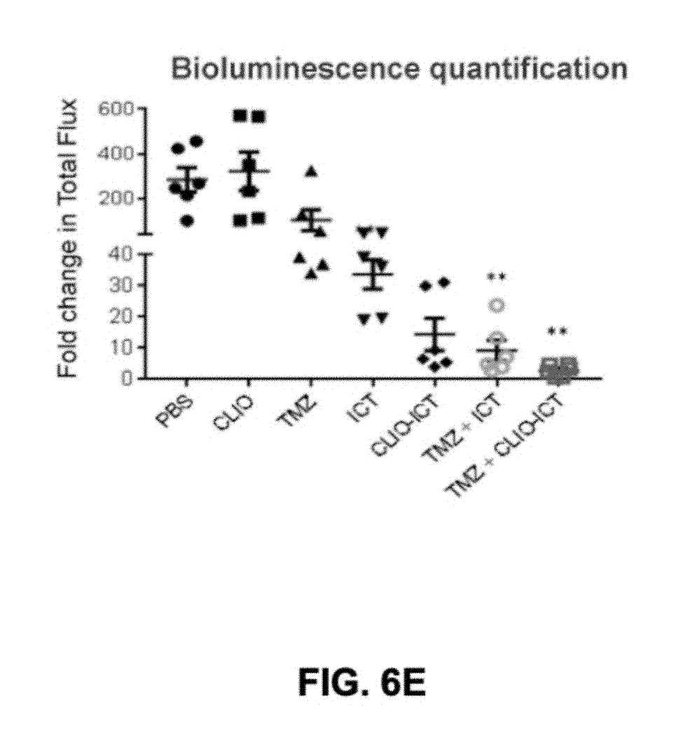

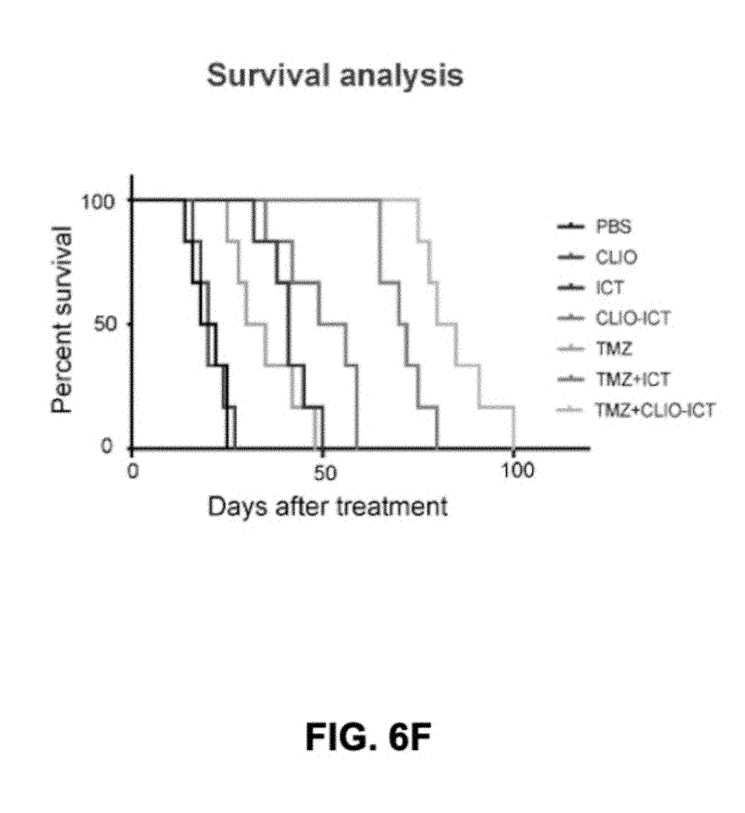

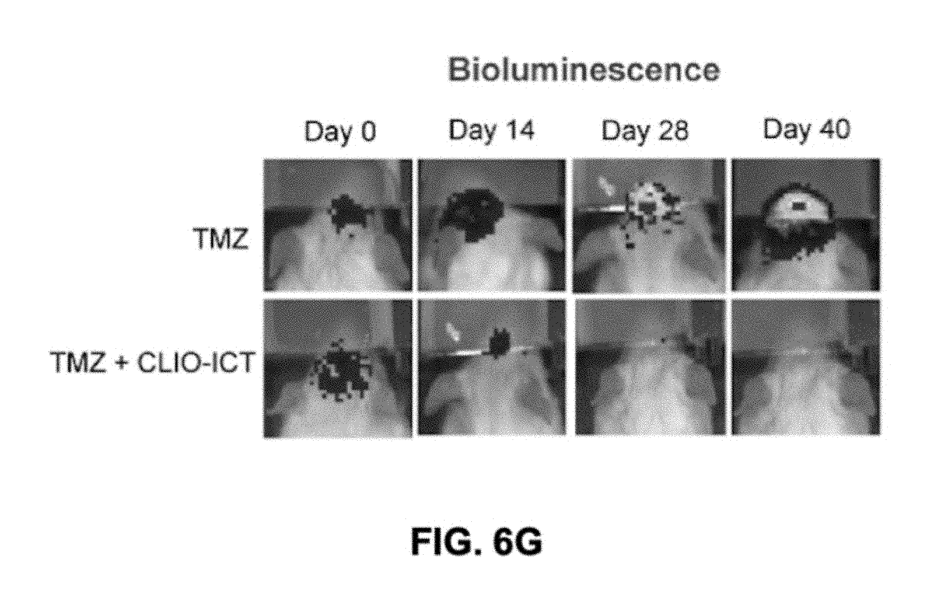

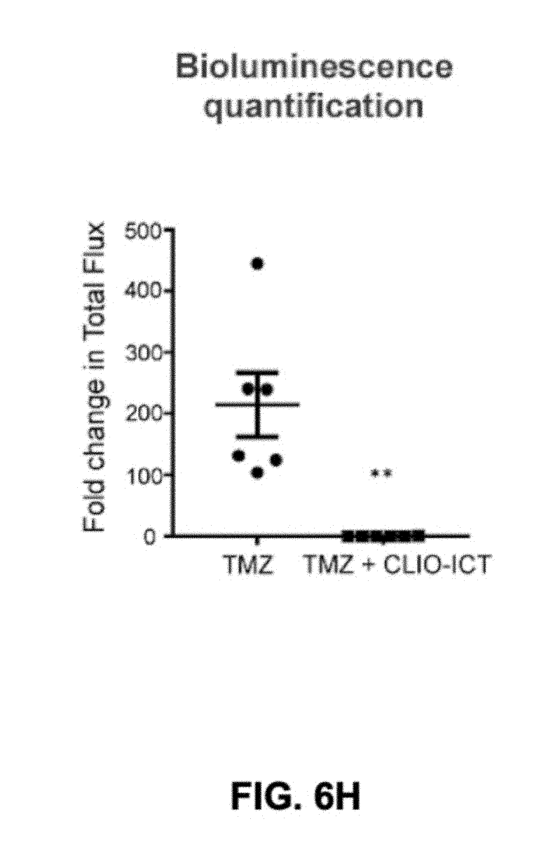

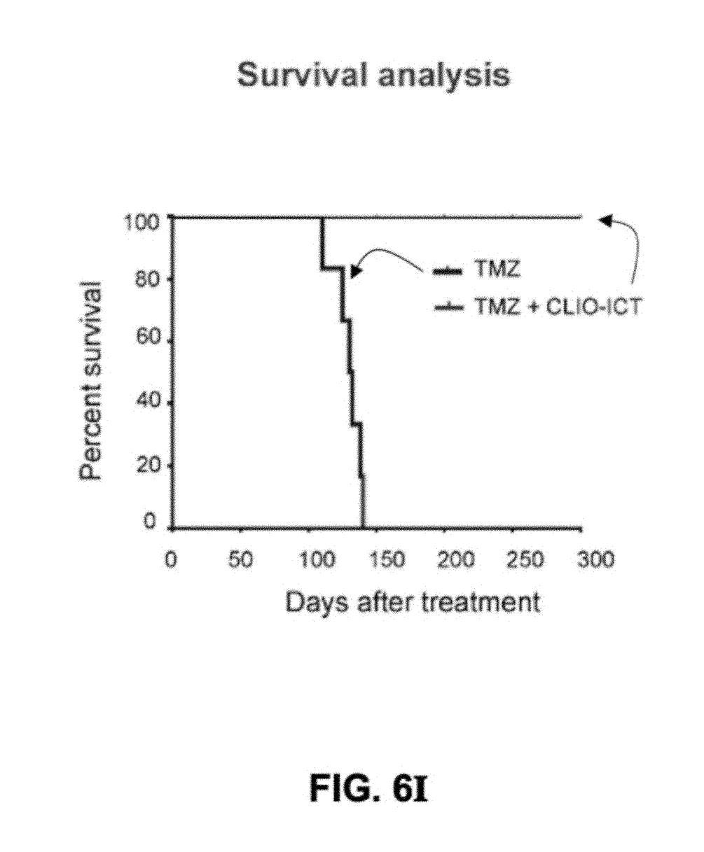

FIG. 6A-I show CLIO-ICTs increase the antitumor efficacy of temozolomide (TMZ) in vivo. FIG. 6A shows in vitro analysis of temozolomide chemosensitivity by cell viability assay. Panel of GBM cells were exposed to increasing doses of temozolomide (0-500 mmol/L) for 72 hours and cell viability was calculated by MTS assay. For in vivo experiments, tumors were initiated with orthotopic injections of pcGBM39 cells (FIGS. 6B-F) and pcGBM2 cells (FIGS. 6G-I). Mice were treated with TMZ alone (200 mg/kg) or in combination with CLIO-ICT (0.5 mmol Fe/kg and 80 mg/kg of ICT) and ICT (80 mg/kg of ICT), CLIO (0.5 mmol Fe/kg), or vehicle.

FIG. 6B shows representative T₂-weighted MR images of mice brain. T₂ FSE sequences were used to capture coronal T₂-weighted images. Yellow dotted line represents tumor periphery.

FIG. 6C shows quantification of tumor volumes before and after treatment. T₂-weighted MR scans were used to calculate tumor volumes using Osirix software. In FIGS. 6F, 6I the Kaplan-Meier survival curves of control and treated mice (n=6, pcGBM39; n=6, pcGBM2) demonstrate a significant survival benefit of CLIO-ICT and ICT in combination with TMZ as compared with vehicle, log-rank Mantel-Cox test Bioluminescence in vivo images of tumors in control and treated mice in pcGBM39 (FIG. 6D) and pcGBM2 (FIG. 6G) tumor models. FIG. 6E and FIG. 6H show quantification of the bioluminescent signals. Fold change in total flux represents the ratio: total flux after treatment/total flux before treatment.

The method according to embodiments of the invention are a glioblastoma treatment method with the step of administering to a subject in an effective amount theranostic cross-linked nanoparticles which are conjugated to a vascular disrupting agent (ICT) and secured with a matrix-metalloproteinase cleavable peptide. The matrix-metalloproteinase (MMP) activatable conjugate could be described, with in one embodiment lacking folate, as a vascular disrupting agent which binds to the colchicine binding site of tubulin. The vascular disrupting agent could be selected from the group consisting of azademethylcolchicine, azacolchicine, N-methyl desacetylcolchicine, desacetylcolchicine, and N-acetylcolchinol-O-phosphate. The matrix-metalloproteinase cleavable peptide comprises in various embodiments the amino acid sequences as described in U.S. application Ser. No. 14/908,096 filed on Jan. 27, 2016. These sequences are hereby incorporated by reference. The nanoparticles comprise a magnetic resonance imaging (MRI) contrast agent. The contrast agent comprises an iron selected from the group consisting of gadolinium, iron, platinum, manganese, copper, gold and barium. The MRI contrast agent comprises an iron selected from the group consisting of iron oxides, magnetide (Fe3O4) and maghemite (Fe2O3). The iron oxide could be superparamagnetic iron oxide (SPIO) or ultra-small superparamagnetic iron oxide (USPIO).

What is claimed is:

1. A glioblastoma treatment method, comprising the step of administering to a subject in an effective amount theranostic cross-linked iron oxide nanoparticles (CLIO) which are conjugated to a vascular disrupting agent (ICT) and secured with a matrix-metalloproteinase cleavable peptide.

2. The method as set forth in claim 1, wherein the matrix-metalloproteinase cleavable peptide lacks folate.

3. The method as set forth in claim 1, further comprising the step of monitoring the delivery of the administered effective amount with magnetic resonance imaging (MRI).
